(19) United States
(12) Patent Application Publication
(10) Pub. No.: US 2009/0043272 A1
(43) Pub. Date: Feb. 12, 2009

(54) TRAINING PANT ARTICLE AND METHOD FOR MANUFACTURING

(75) Inventors: DANE JACKSON, NEW HANOVER, PA (US); MARIELA MUNIZ, WEST CHESTER, PA (US)

Correspondence Address: AMSTER, ROTHSTEIN & EBENSTEIN LLP, 90 PARK AVENUE, NEW YORK, NY, 10016, US

(73) Assignee: TYCO HEALTHCARE RETAIL SERVICES AG, Schaffhausen (CH)

(21) Appl. No.: 11/835,459

(22) Filed: Aug. 8, 2007

(51) Int. Cl. A61F 13/15; G09B 23/00

(52) U.S. Cl. 604/367; 434/262; 604/385.01; 604/385.25

(57) ABSTRACT

A method for projecting leakage performance for an absorbent article includes applying a liquid insult to a first sample article on a testing apparatus and recording leakage, applying a liquid Insult to a second sample article on the testing apparatus and recording leakage, and calculating a projected frequency of actual leakage for the absorbent article according to a linear regression formula. A method of designing an absorbent article includes determining a maximum acceptable frequency of overnight leakage for the article, calculating a target Static Retention Index, testing the absorbent article to determine an average mannequin leakage value, and modifying the absorbent article structure to adjust the average test mannequin leakage value until the average test mannequin leakage value is substantially equal to the target Static Retention Index. A training pant includes a core having a Static Retention Index of less than about 1.4 g.

### FIELD OF THE INVENTION

The invention relates generally to absorbent articles, and in particular to a training pant and method for designing training pants based on a liquid retention index.

### BACKGROUND OF THE INVENTION

The toilet training process for many children can be a difficult period of transition. A major challenge is overcoming the child's dependency on disposable or cloth diapers. Manufacturers of absorbent articles have developed “training pants” to help children transition between diapers and underwear. Training pants are similar to disposable diapers, in that they contain a limited amount of absorbent material. The absorbent material is Intended to capture liquids in the event the child has an “accident”. Unlike diapers, which are taped or pinned around the body, training pants are designed to be pulled up or down like underwear, so that the child can remove the training pants during toilet training.

Training pants are generally not secured or sealed as tightly to the wearer as diapers. In addition, the core is not designed to absorb and retain a large volume of liquid. As a result, training pants may be subject to a small amount of leakage when a child has an accident. While a certain amount of bed wetting may be expected from training pants, it is desirable to maintain the amount of bed wetting within an acceptable level. Unfortunately, there has been no reliable or consistent method for predicting overnight leakage and designing a training pant that controls leakage. Too often, training pants are designed without an accurate prediction of leakage. Rather than predict leakage, designers may simply add absorbent material and other materials to the training pant in the hopes of controlling leakage. This can result in an “overdesigned” training pant, with excess absorbent materials or other product components that either overcompensate for the actual sources of leakage, or fall to address the sources of leakage.

### SUMMARY OF THE INVENTION

The drawbacks of known training pants and manufacturing methods are resolved in many ways by a training pant, and methods for testing and designing training pants, all in accordance with the invention as described herein.

In a first exemplary embodiment of the invention, a method for projecting actual leakage performance for an absorbent article includes the steps of placing a first sample of the absorbent article on a testing apparatus in a first orientation, applying a first insult of liquid to the first sample on the testing apparatus, recording the leakage of liquid in the first orientation, placing a second sample of the absorbent article on the testing apparatus in a second orientation different from the first orientation, applying a second insult of liquid to the second sample on the testing apparatus, recording the leakage of liquid in the second orientation and calculating a projected frequency of actual leakage for the absorbent article according to a linear regression formula.

In a second exemplary embodiment of the Invention, a method of designing an absorbent article includes the steps of determining a maximum acceptable frequency of overnight leakage for the article, calculating a target Static Retention Index based on the maximum acceptable frequency of overnight leakage, testing an absorbent article design in a plurality of different orientations that simulate positions to determine mannequin leakage values in the different orientations, calculating the average mannequin leakage value based on leakage recorded in the plurality of different orientations, comparing the average mannequin leakage value to the target Static Retention Index, and modifying the absorbent article design to adjust the average test mannequin leakage value until the average test mannequin leakage value is substantially equal to the target Static Retention Index.

In a third exemplary embodiment of the invention, a training pant includes an outer cover, an inner liner for positioning against a wearer and an absorbent core between the outer cover and inner liner, the core comprising a Static Retention Index of less than about 1.4 g.

### BRIEF DESCRIPTION OF THE DRAWINGS

The foregoing summary and the following description will be better understood in conjunction with the drawing figures of which,

FIG. 1 is a perspective view of an exemplary embodiment of a training pant in accordance with the present invention;

FIG. 2 is a top plan view of the training pant of FIG. 1, with the training pant in an opened condition; and

FIG. 3 is a sample plot illustrating a relationship between performance data that is used in accordance with the present invention.

### DETAILED DESCRIPTION OF THE PREFERRED EMBODIMENTS

Although the invention is illustrated and described herein with reference to specific embodiments, the invention is not intended to be limited to the details shown. Rather, various modifications may be made in the details within the scope and range of equivalents of the claims and without departing from the invention.

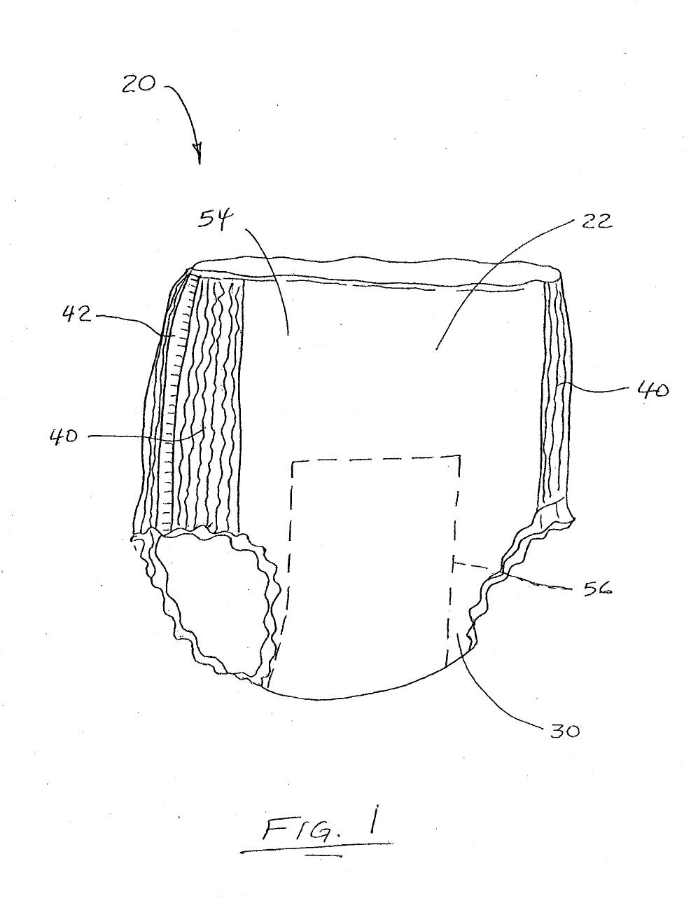

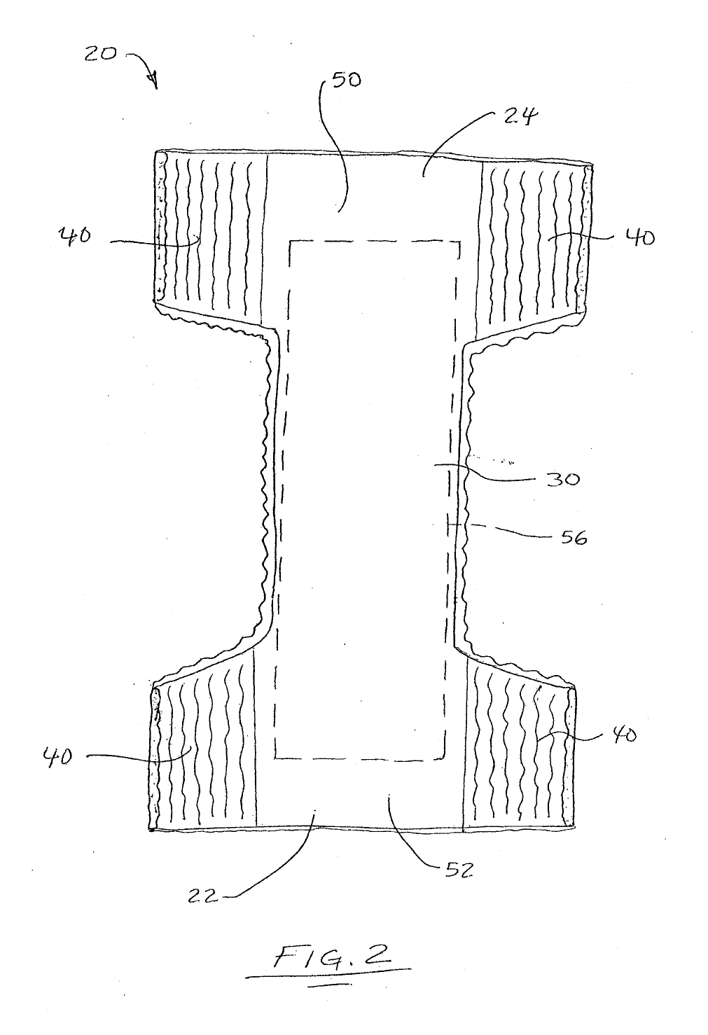

Referring to FIGS. 1 and 2, a training pant 20 in accordance with the invention is shown. Training pant 20 includes a front section 22, a rear section 24 and a crotch section 30 connecting the front section with the rear section. Front and rear sections 22, 24 each have a pair of stretchable panels or ears 40. Ears 40 on the front section 22 are connected to ears on the rear section 24 by side seams 42. The training pant is delivered to the consumer in the closed configuration shown in FIG. 1. In this configuration, the training pant can be pulled up onto the child, similar to conventional underpants. The side seams 42 are designed to be separated easily to remove the training pant from the child.

A body portion 50 extends along the center of the training pant. Preferably, the body portion 50 extends along the entire length of the training pant 20, beginning at the front section 22 through the crotch section 30 to the rear section 24. Body portion 50 includes one or more layers of material. In the preferred embodiment, body portion 50 includes a inner body-facing liner 52 and an outer layer 54 facing away from the wearer. A core 56 is positioned between inner liner 52 and outer layer 54. Preferably, the core 56 includes absorbent material that absorbs and retains liquid. For example, the core 56 may include a matrix formed of pulp with or without a super absorbent polymer dispersed throughout the matrix.

The performance of training pant products, such as that shown in FIGS. 1 and 2, can be influenced by a number of design parameters, Including but not limited to the selection of materials used in the core, the absorbency of materials used in the core, the materials used in side seams, the materials used in the leg cuffs, and the configuration of the leg cuffs. In a preferred embodiment of the invention, a training pant Includes a dual leg cuff configuration as shown in U.S. Patent Application Publication No. 2007/0073259-A1, the contents of which are incorporated by reference herein. Although the foregoing parameters influence performance, the selection and combination of materials to construct a training pant typically does not correlate well with the overnight leakage performance of the training pant. For example, a training pant that includes a super absorbent polymer core and one cuff or gasket in each leg opening may still allow an undesirable volume of leakage. Providing additional super absorbent polymer may not improve overnight leakage performance. A low incidence of leakage on a particular training pant could be attributed to materials used in the core in combination with other parameters that are more difficult to quantify. Therefore, training pant performance can not be accurately predicted based solely on the materials selected.

The volume and frequency of leakage observed for children wearing training pants while awake has been found to have little or no correlation to leakage observed in children wearing the training pant while asleep. One factor is that the child is learning to use the toilet and will try to avoid insulting the training pant when they are awake. Another factor is believed to be that children who are awake are typically standing or sitting upright, with the absorbent portion of the training pant in position to receive the insult by gravity. Children who are asleep, in contrast, are usually lying prone or to one side, which tends to allow liquid to bypass the absorbent portion and flow directly to other areas, such as a leg opening or waist opening. Moreover, a child who is awake and insults the training pant is more likely to be moving, which distributes the insult more evenly Into the absorbent materials of the pant and decreases the likelihood of leakage. The sleeping child is more likely to be lying motionless, such that liquid channels through the training pant and does not get distributed evenly. Because of these factors, the performance of a training pant under “dynamic” conditions can not be used to accurately predict the overnight leakage performance of training pants, which occurs under conditions that are more or less “static.”

It has been discovered that the frequency of overnight leakage of a training pant is not merely a function of the selection of materials used, but more a function of the product's ability to retain liquid under static conditions. Under static conditions during sleep, liquid is not necessarily directed to absorbent parts of the training pant, as noted above. In incidents of overnight leakage, the child may be sleeping generally motionless on their side, back or stomach, with gravity having a primary influence on the direction of liquid migration. Often times, the insult can be directed by gravity toward the side seam or waist band of the training pant, and away from the crotch area where the absorbent core is positioned. This may direct liquids to more leakage-prone areas of the pant. As a result, the absorbency of the core has less of an influence on leakage than other factors.

A training pant's ability to retain liquid can generally be quantified in terms of a Static Retention Index, or “SRI”. SRI measures the amount of leakage from a training pant after a first insult is applied to the training pant. Leakage is measured with the training pant configured in a number of orientations that may simulate the training pant being worn in different positions. For example, leakage may be measured with the training pant in an orientation that simulates the training pant being worn while the wearer is sleeping on his or her back, stomach or side. Leakage measurements are then combined to calculate the SRI for the training pant. Using a predetermined relationship between SRI and frequency of overnight leakage, the actual performance of the training pant design can be predicted accurately, without the use of consumer studies or surveys.

It has been discovered that overnight leakage performance of a training pant design can be accurately predicted by correlating actual overnight leakage with SRI values obtained from tests performed on mannequins. This predictive correlation allows a training pant product to be designed based on a pre-determined or “target” mannequin leakage. Because the design process is guided by mannequin testing, as opposed to actual overnight leakage performance, the source of leakage on the training pant can be easily identified, and modifications to that area of the training pant can be made. The time and expense associated with actual leakage performance testing, including consumer tests and surveys, are reduced or eliminated. In addition, the focus on SRI greatly reduces the potential for selecting too much super absorbent polymer or other material in order to overcompensate for deficiencies, which often occurs when the design process is dictated solely by choice and amount of material. The ability to accurately predict actual product performance through mannequin testing, in conjunction with the ability to identify the specific locations of leakage during that testing, has resulted in the development of a training pant with superior overnight leakage performance, without overdesign.

The frequency of actual overnight leakage of a training pant, measured as a percentage, can be predicted based on linear regression analysis. Data collected from a series of mannequin tests and actual overnight performance tests can be correlated with one another and plotted in a straight line. The horizontal or “X” axis corresponds to a first insult mannequin leakage, measured in grams. The vertical or “Y” axis corresponds to in-use leakage as a volume percentage of insult. The straight line “L” depicts a line of best fit for data points. Line “L” is characterized by the following relationship:

Lₐ=α+(β×SRI),

where Lₐ is equal to the percent frequency of actual overnight leakage, and SRI is equal to average leakage in grams based on first insult mannequin leakage measurements taken with the training pant tested in a number of orientations.

The correlation between actual overnight leakage and first insult mannequin leakage was demonstrated in a study which used data from first insult mannequin leakage tests (“mannequin tests”) and data from actual leakage performance tests (“in-use tests”). In the mannequin tests, a mannequin was selected with dimensions that correspond to the waist, buttocks and thigh regions of a toddler. The mannequin was connected to a test apparatus that administers liquid insult to a diaper, training pant or other garment being tested.

A test apparatus and mannequin manufactured by Marketing Technology Services (MTS), Inc. were used in the experiments described herein. Specifically, an MTS TEFO test apparatus, MTS Super Toddler mannequin, and MTS software were used to perform the tests. The mannequin had a waist dimension of 58 cm and a thigh dimension of 33 cm.

In performing the mannequin tests, a 1% saline solution at room temperature was prepared. The solution was placed in a reservoir in the test apparatus. Power was turned on, and the manufacturer's procedures for calibrating volume and flow rate were completed. A training pant was selected for testing, and the dry weight of the training pant was recorded. The training pant was then fitted around the mannequin in the manner that it would be worn during actual use. The mannequin was oriented to simulate the position of a wearer lying on their left side.

A sample of 1% saline solution was prepared for use as the liquid insult and poured into the reservoir of the test apparatus. An empty drip pan was weighed and placed beneath the mannequin. The motor of the test apparatus was then started to introduce liquid from the reservoir to the training pant to simulate a liquid insult. The test apparatus was programmed to deliver 100 ml of the saline solution at a flow rate of 7 ml/sec from a crotch region on the mannequin. The mannequin and training pant were monitored for visible signs of leakage, and the location of any leakage (e.g. the leg cuff or side seam of the training pant) was recorded. When all leakage was recovered from the training pant, the drip pan was removed and weighed to determine the volume of leakage. The test was then repeated with the mannequin oriented to simulate the position of a wearer lying on their right side. The leakage measurements from the mannequin lying on left and right sides were then averaged to determine an SRI value.

The following section summarizes the test procedure that was applied to the training pant using the MTS TEFO test apparatus, MTS Super Toddler mannequin, and MTS software.

### Mannequin Test

### 1. Scope

To measure leakage performance of diapers and training pants.

### 2. Safety and Handling Precautions

General safe laboratory practices—refer to MSDS and equipment manuals.

### 3. Equipment and Materials

3.1 MTS TEFO Tester
3.2 MTS Mannequin Doll

| Training Pants | |
|---|---|
| Medium (2T-3T) | Super Toddler |
| Large (3T-4T) | Super Toddler |
| X-Large (4T-5T) | Super Toddler |

3.3 1% NaCl Solution in water
3.4 Digital Balance with at least 100 g capacity and 0.1 g resolution (or equivalent).
3.5 Digital Balance with at least 5000 g capacity and 0.1 g resolution (or equivalent).
3.6 Drip pan
3.7 Scissors

### 4. Solution Preparation

4.1 Place 5-liter pitcher on the balance and tare.
4.2 Add 4950 g of deionized water to the pitcher and remove from the balance.
4.3 In a plastic weighing dish, weigh out 50 g of NaCl.
4.4 Add the NaCl to the pitcher and stir until the NaCl is dissolved.
4.5 Check the salinity of the solution using the refractometer.
4.6 Fill liquid reservoir of MFS TEFO Tester.

### 5. Test Preparation

5.1 Turn Power box on.
5.2 Turn computer, monitor and both balances (scale 1 & scale 2) on.
5.3 Go to: START>PROGRAMS>ROCKWELL SOFTWARE>RSView32>RSView32 RUNTIME
5.4 Once in the RSView32 Runtime screen, go to: FILE>OPEN>mts_{—}2.rsv) RUN PROJECT bar
5.5 Maximize screen. Before clicking on “BABY MANNEQUIN”, all other valves are turned OFF. Open all other test methods and verify that the VALVE button is off. (Valve off=RED).
5.6 Click on “BABY MANNEQUIN”.
5.7 You are now in the Mannequin Menu. Click on the CALIBRATE button in order to calibrate volume and flow rate.
5.8 CALIBRATION (NOTE: Calibration is done before the testing is run and/or when the volume, flow rate, or device is changed).
5.8.1. There are five steps in the CALIBRATE ABSORPTION/ACQUISITION screen:
   Step #1—Flow Rate—Select 7 ml/sec
   Step #2—Volume—Enter desired volume. Hit Enter and Click on the SCALE DATA button.

| | ml of test solution for |
|---|---|
| Product Size | each insult |
| Training Pants-All sizes | 100 |

Step #3—Start Motor—Place plastic container on scale 1. Click on ZERO MOTOR, TARE SCALE (Make sure the scale reads zero), Place plastic container under mannequin, Click on START MOTOR.
Step #4—Calibrate—After insult, place plastic container back on scale 1. Type the recorded ACTUAL MILIMETERS on the space provided. Hit Enter and Click on the CALIBRATE button.
   Note: If the recorded ACTUAL MILLIMETERS doesn't read the desired volume, Steps 3&4 is repeated until desired volume is obtained.
   If ACTUAL MILLIMETERS does read the desire volume, the apparatus is calibrated. DO NOT type insult weight, simply move to Step #5.
Step #5—Return to test—Once the apparatus is calibrated, select BABY MANNEQUIN TEST to run and begin testing under the selected calibrated condition.

### 6. Test Procedure

6.1 SAMPLE PREPARATION
6.1.1 Weigh and record product.
6.1.2 Carefully fit weighted product on mannequin.
6.1.3 Choose desire mannequin position
   6.1.3.1 Lying on side (Right or Left)
   6.1.3.2 Lying on back
   6.1.3.3 Lying on stomach
   6.1.3.4 Standing
      Note: Different mannequin positions can be obtained by simply adjusting the latch located on the stem of the mannequin stand.
   6.1.4 Once sample is prepared:
      6.1.4.1 Enter time delay between liquid insults. Since time is recorded in seconds, type 300.
      6.1.4.2 Enter number of doses (3).
      6.1.4.3 Enter dry weight and sample ID.
      6.1.4.4 Place the dripping pan on scale 2.
      6.1.4.5 Click on AUTO MANNEQUIN/RESET, ZERO MOTOR, TARE SCALE (make sure the scale reads zero). Remove dripping pan from scale and place it underneath mannequin.
      6.1.4.6 Click on START MOTOR.
      6.1.4.7 Monitor mannequin and look for any sign of leakage. If leakage occurred, note leakage location (ex: over leg, through side seam, etc).
      6.1.4.8 When all leakage has been recovered by the dripping pan, place it on scale 2 and record leakage weight.
      6.1.4.9 Wait until the timer goes back to zero (after five minutes) and repeat set 6.1.4.5.
      6.1.4.10 Remove sample from mannequin (scissors may be used). Make sure mannequin is dry before fitting the next sample.

### 7. Report

7.1 Leakage after each insult
7.2 Total Leakage
7.3 Leakage location on sample

The same training pant was also subjected to actual overnight leakage testing in home use tests, Data was collected from 200 interviews with parents of children who wore samples of the training pant overnight. Children weighing between 32 and 40 pounds were fitted with training pants, and their parents were surveyed to determine whether any overnight leakage through the training pant was observed.

Four different training pant designs, Examples A through D, were subjected to first insult mannequin tests and in-use leakage tests. The data collected for Examples A through D are summarized in the table below:

| Test Sample | SRI (g) | In-Use Leakage, Lₐ (%) |
|---|---|---|
| A | 1.58 | 8.5 |
| B | 2.39 | 10.9 |
| C | 0.46 | 6.7 |
| D | 0.22 | 6.0 |

The percentage of in-use leakage for each of the samples is presented in the third column of the table. The in-use leakage percentage was equal to the percentage of all training pants tested for which leakage was observed. The test data shows a direct relationship between the SRI and the frequency of actual in-use leakage, Lₐ. Test samples that showed a lower first insult mannequin leakage also showed a lower frequency of in-use leakage. Conversely, test samples that showed a higher first insult mannequin leakage also showed a higher frequency of in-use leakage.

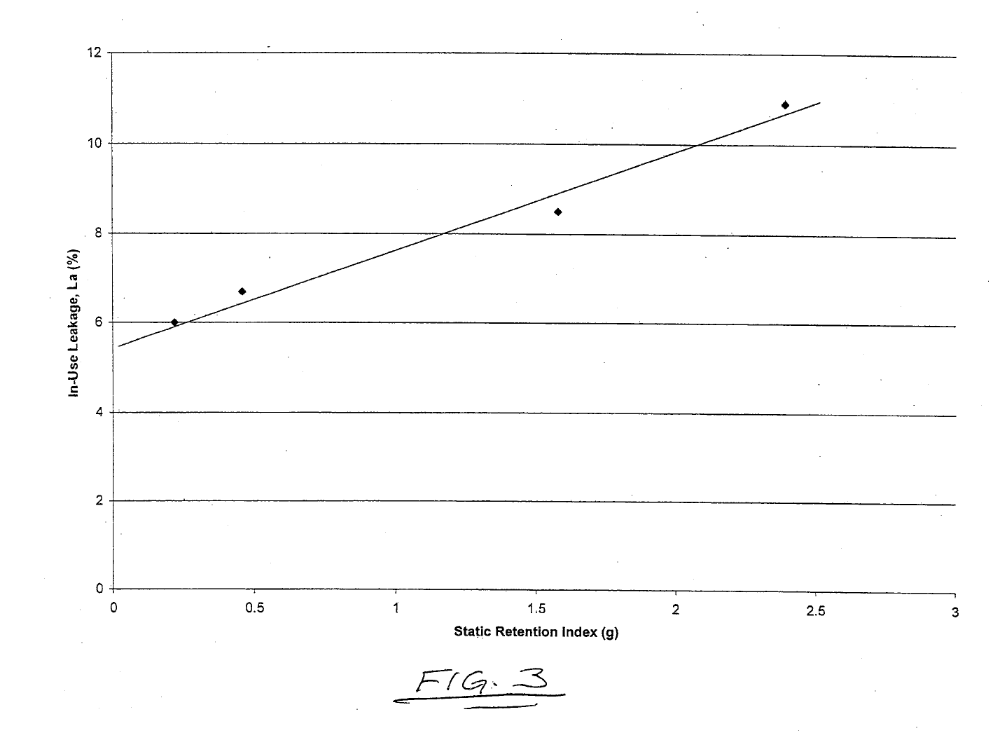

The data for test samples A-D are plotted in FIG. 3. The plot reflects a generally linear relationship between first Insult mannequin leakage and frequency of in-use leakage. Using regression analysis, an equation represented by a line of best fit between the data points is:

Lₐ=5.53+2.14(SRI)

where once again, Lₐ is equal to the percent frequency of actual overnight leakage, and the SRI is an average leakage in grams based on first Insult mannequin leakage measurements taken with the training pant tested in a number of orientations.

The relationship described above between SRI and frequency of actual overnight leakage can be applied to predict the performance of a specific training pant product. For example, a proposed product can be subjected to mannequin testing to determine an SRI for the proposed product. Using the established relationship between SRI and the frequency of actual overnight leakage (e.g. the plot in FIG. 3 and equation), a frequency of actual overnight leakage can be predicted for the product.

Applicants have found that this relationship can be turned around and used to adjust the performance of a training pant to a desired level. In such a case, a maximum acceptable frequency of overnight leakage (MFOL) is determined. Once MFOL is established, a target SRI (SRIₜ) is calculated using the following equation derived from the linear regression equation above, solving for SRIₜ:

SRIₜ=Ω(MFOL)−ω;

SRIₜ is the target SRI in grams, and represents a maximum desirable SRI value for the product. MFOL is the maximum acceptable overnight leakage value (%). The variable Ψ is preferably between about 0.4 and 0.5, and ω is preferably between about 2 and 3.

Once a value for SRIₜ is determined from the equation, a product can be designed and tested to determine whether it has an SRI that is equal to or less than the SRIₜ value. If the test indicates an SRI that is greater than SRIₜ, the predicted frequency of overnight leakage will exceed MFOL, indicating a need or desire to modify the product. Because the product is observed on a mannequin, the locations of leakage can be visually identified, providing an indication of where the product must be modified. Moreover, the relative difference between the SRI value and SRIₜ will provide an approximate indication of how much modification needs to be made. For example, a relatively small difference between the SRI value and SRIₜ may indicate the need to make a minor change to a leg cuff, for example, whereas a larger difference may indicate the need to alter the amount and type of materials used.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. A method for projecting actual leakage performance for an absorbent article comprising the steps of:
placing a first sample of the absorbent article on a testing apparatus in a first orientation;
applying a first insult of liquid to the first sample on the testing apparatus;
recording leakage of the liquid applied to the first sample in the first orientation;
placing a second sample of the absorbent article on the testing apparatus in a second orientation different from the first orientation;
applying a second insult of liquid to the second sample on the testing apparatus;
recording leakage of the liquid applied to the second sample in the second orientation; and
calculating a projected frequency of actual leakage for the absorbent article according to the linear regression formula:
Lₐ=α+(β×SRI)
wherein,
Lₐ actual projected leakage (%);
α is between about 5 and about 6;
β is between about 1.5 and about 2.5; and
SRI=an average of the leakage recorded in the first orientation and the leakage recorded in the second orientation.

2. The method of claim 1 wherein α is about 5.53.

3. The method of claim 1 wherein β is about 2.14.

4. The method of claim 1 wherein the sample of the absorbent article is placed on the testing apparatus with a left side portion facing upwardly in the first orientation.

5. The method of claim 1 wherein the sample of the absorbent article is placed on the testing apparatus with a right side portion facing upwardly in the first orientation.

6. A method of designing an absorbent article comprising the steps of:
determining a maximum acceptable frequency of overnight leakage value for the article;
calculating a target Static Retention Index based on the maximum acceptable overnight leakage value according to the formula:
SRIₜ=Ψ(MFOL)−ω;
testing an absorbent article design in a plurality of different orientations that simulate positions to determine mannequin leakage values in said plurality of different orientations;
calculating an average mannequin leakage value based on leakage recorded in the plurality of different orientations;
comparing the average mannequin leakage value to the target Static Retention Index;
modifying the absorbent article design to adjust the average mannequin leakage value until the average mannequin leakage value is substantially equal to the target Static Retention Index,
wherein
SRIₜ=target Static Retention Index (g);
MFOL=maximum acceptable frequency of overnight leakage (%);
Ψ is between about 0.4 and 0.5; and
ω is between about 2 and 3.

7. The method of designing an absorbent article of claim 6 wherein Ψ is about 0.46.

8. The method of designing an absorbent article of claim 6 wherein ω is about 2.6.

9. The method of designing an absorbent article of claim 6 wherein the step of modifying the absorbent article design comprises the step of Identifying an area on the absorbent article design where leakage occurs and modifying that area of the absorbent article design.

10. The method of designing an absorbent article of claim 6, wherein the step of modifying the absorbent article design comprises the step of changing a leg cuff configuration.

11. The method of designing an absorbent article of claim 6, wherein the step of modifying the absorbent article design comprises the step of adding or subtracting an amount of super absorbent polymer in a core.

12. The method of designing an absorbent article of claim 6, wherein the step of modifying the absorbent article design comprises the step of adding or subtracting an amount of absorbent pulp in a core.

13. The method of designing an absorbent article of claim 6, wherein the step of modifying the absorbent article design comprises the steps of changing a leg cuff configuration and adding or subtracting an amount of super absorbent polymer in a core.

14. The method of designing an absorbent article of claim 6, wherein the step of modifying the absorbent article design comprises the steps of changing a leg cuff configuration and adding or subtracting an amount of absorbent pulp in a core.

15. A training pant comprising an outer cover, an inner liner for positioning against a wearer, and an absorbent core interposed between the outer cover and inner liner, the training pant having a Static Retention Index of less than about 1.4 g.

16. The training pant of claim 15 wherein the Static Retention Index is less than about 1.2 g.

17. The training pant of claim 15 wherein the Static Retention Index is less than about 1.0 g.

18. The training pant of claim 15 wherein the Static Retention Index is less than about 0.8 g.

19. The training pant of claim 15 wherein the absorbent core comprises a super absorbent polymer.

20. The training pant of claim 15 further comprising a leg cuff.

21. The training pant of claim 15 further comprising a dual leg cuff.

22. The training pant of claim 15 comprising a matrix formed of pulp with a super absorbent polymer dispersed in at least a portion of the matrix.

23. The training pant of claim 15 comprising a matrix formed of pulp without a super absorbent polymer.

24. The training pant of claim 15 comprising a side seam adapted to be separated to open the training pant.
